(19) United States
(12) Patent Application Publication
(10) Pub. No.: US 2018/0319762 A1
(43) Pub. Date: Nov. 8, 2018

(54) 5-deoxy-irilin B Having Angiotensin-I-converting enzyme Inhibition Activity derived from Salicornia SPP. and Composition Containing the Same

(71) Applicant: PHYTO CORPORATION, Seoul (KR)

(72) Inventors: Deuk Hoi KIM, Ilsandong-gu, Gyeonggi-do (KR); Young Kyu YANG, Seoul (KR); Gang Deog LEE, Suwon-si Gyeonggi-do (KR); Eun Ah CHO, Seoul (KR); Da Young HONG, Seoul (KR); Yun Kyoung HA, Incheon (KR); Mee Hyang KWEON, Seoul (KR)

(21) Appl. No.: 15/773,049

(22) PCT Filed: Nov. 4, 2016
(86) PCT No.: PCT/KR2016/012624

§ 371 Date: May 2, 2018

(30) Foreign Application Priority Data
Nov. 4, 2015 (KR) 10-2015-0154424
Nov. 3, 2016 (KR) 10-2016-0145862

(51) Int. Cl. C07D 311/40; A23L 23/00; A23L 33/105; A61K 36/21; C07D 311/36; A61P 9/12; A61K 9/00

(52) U.S. Cl. CPC C07D 311/40; A23L 23/00; A23L 33/105; A61K 36/21; C07D 311/36; A61P 9/12; A61K 2236/51; A23V 2002/00; A61K 2236/55; A61K 2236/39; A61K 2236/37; A61K 2236/15; A61K 2236/13; A61K 9/0056

(57) ABSTRACT

A compound having antihypertensive activity, especially 5-deoxy-irilin B having angiotensin-I-converting enzyme inhibition activity, derived from Salicornia SPP., a salty sauce containing the same, and a composition for the prevention and treatment of hypertension containing the same are provided. The Salicornia SPP.-derived salty sauce having superior sensory and functional properties can be produced through cutting or grinding and then hydrolyzing of Salicornia SPP. alone, thus increasing the amounts of polyphenols, which is effective in the treatment of hypertension, and glutamic acid, which is responsible for a savory taste; through purification, thus improving sensory properties such as removal of unpleasant tastes and odors and decoloration; and through concentration under reduced pressure, thus exhibiting both a savory taste and a salty taste, and can also be efficiently utilized as functional foods and medications for the prevention of hypertension and cardiovascular disease, and contains large amounts of organic nutrients and is thus nutritionally useful.

### TECHNICAL FIELD

The present invention relates to a compound having antihypertensive activity derived from Salicornia SPP. and, more particularly, to 5-deoxy-irilin B having angiotensin-I-converting enzyme (ACE) inhibition activity derived from Salicornia SPP., a salty sauce containing the same, and a composition for the prevention and treatment of hypertension containing the same.

### BACKGROUND ART

Salicornia SPP. is a halophyte that grows in very salty soil, and lives in areas around tidal beaches, reclaimed land, salterns, etc., which have high salt concentration in the soil such that general terrestrial plants cannot proliferate. Salicornia SPP. is distributed throughout the world, and communities thereof are particularly well developed in the areas of the west and south coasts of Korea.

Salicornia SPP., the cells of which have high salt content, tastes very salty when ingested, and absorbs various minerals and microelements, including not only sodium (Na) but also potassium (K), calcium (Ca), magnesium (Mg), iron (Fe), phosphorus (P) and the like, which are dissolved in sea water and thus accumulate therein. Furthermore, it contains large amounts of organic nutrients, such as amino acids, enzymes, dietary fiber, etc., and its nutritional value is very high. Salicornia SPP. is known to have a variety of physiological effects, such as antihypertensive effects, antidiabetic effects, anticholesterol effects, melanogenesis inhibition effects, and antioxidation effects.

Salicornia SPP. contains sodium (Na), potassium (K) and the like and thus has a salty taste, and also contains amino acid, such as glutamic acid, aspartic acid and the like, which exhibit a savory taste, whereby it may be utilized as a seasoning sauce via a purification process.

A seasoning sauce is used to enhance the flavor of a dish during cooking. To this end, appropriate amounts of salty and savory tastes are required. However, since a conventional seasoning sauce includes a large amount of salt such as refined salt or solar salt, health problems may occur due to the excessive intake of salt.

Korean Patent Application Publication Nos. 10-2012-0133868 (Composition and method for preparing a sauce of grilled meat and fish using Salicornia SPP.) and 10-2007-0109798 (Method of manufacturing a soy sauce of abalone using Salicornia SPP. and soy sauce of abalone made thereby) disclose a method of mixing some of a Salicornia SPP. extract with a soy sauce and other sauces. However, these patents are disadvantageous because the amount of the active ingredient derived from Salicornia SPP. is limited and such sauces are not composed exclusively of Salicornia SPP. and have no sensory or functional properties.

Korean Patent Application Publication No. 10-2007-0048077 discloses a salt replacement and a method of producing the same, wherein the salt replacement is produced by subjecting Salicornia SPP. to extraction, centrifugation and ultrafiltration to obtain a filtrate that is then dried. However, the product thus obtained is a salt replacement in a solid phase resulting from a drying process, and suffers from an unpleasant taste and unpleasant odor because a purification process is not conducted and also from high production costs due to the use of ultrafiltration. Furthermore, considerable amounts of nutrients and functional components are lost during the filtration process such as ultrafiltration.

Korean Patent Application Publication No. 10-2001-0083037 discloses a liquefied plant salt and a method of producing the same, wherein the liquefied plant salt is obtained by extracting and compressing halophytes. However, the product thus obtained has an unpleasant taste and unpleasant odor due to the absence of a purification process, is very dark, and does not taste sufficiently sweet due to the absence of saccharification via a concentration process, undesirably resulting in very inadequate sensory properties. Moreover, such a liquefied plant salt includes 80 wt % or more of NaCl, and is thus unsuitable for use as a sauce because of the excessively high NaCl content.

With regard to techniques of producing fermented materials using Salicornia SPP., Korean Patent Application Publication No. 10-2011-00936016 discloses a method of fermenting Salicornia SPP. for a long period of time, namely 45 months or more, by the addition of microbial fermentation nutrients, such as an artificial saccharide source such as glucose or sugar, and Korean Patent No. 10-1243361 discloses a method of manufacturing a soy sauce using Salicornia SPP. by fermenting Salicornia SPP. with 35 wt % or more of glucose or sugar for 5 months or more and further adding the fermented liquid with supplementary materials such as garlic, ginger, onion, seaweed, S. zunasi, and other medicinal herbs. However, attempts to develop Salicornia SPP.-derived pure salty sauces composed exclusively of Salicornia SPP., having superior taste and functionality, without the addition of a saccharide source such as sugar or glucose and salt (solar salt or refined salt), have not yet been made.

Meanwhile, hypertension is a major cause of cardio-cerebrovascular diseases such as stroke, myocardial infarction, congestive heart failure, kidney disease and peripheral vascular disease. In Korea, the extent of generation of cerebrovascular disease from hypertension is known to be 35% and the extent of generation of ischemic heart disease from hypertension is known to be 21%. This means that 35% of cases of cerebrovascular disease and 21% of cases of ischemic heart disease could be prevented if the entire population could maintain normal blood pressure. People with high blood pressure tend to experience continued increases in blood pressure, and increased blood pressure increases the risk of cardio-cerebrovascular disease, and persons who have been diagnosed with hypertension require the administration of antihypertensive drugs and improvements in lifestyle.

Many antihypertensive drugs have been developed to treat hypertension, and are classified into, depending on the mechanism of action and the action sites, diuretics, sympathetic nervous system-acting drugs (α,2-adrenergic antagonist, β-adrenergic antagonist), vasodilators, calcium channel blockers, angiotensin-converting enzyme (ACE) inhibitors, etc. Angiotensin-I-converting enzyme (ACE) functions to convert angiotensin-I, which is a decapeptide, into angiotensin-II, which causes vasoconstriction by cutting a dipeptide (His-Leu). An increase in the amount of angiotensin-II that is produced using ACE promotes an increase in blood pressure and the secretion of the anti-diuretic hormone aldosterone and suppresses the emission of water and sodium to thus increase blood circulation, resulting in hypertension. Also, ACE decomposes and deactivates brakykinin, which has a vasodilator action, ultimately increasing the blood pressure. Therefore, the activity of ACE is inhibited, whereby vasoconstriction may be prevented, thus exhibiting the effect of lowering blood pressure, indicating that compounds having ACE inhibition activity may be developed as a medicament for the prevention or treatment of hypertension.

The present inventors have tried to develop salty sauces having improved sensory and functional properties derived from Salicornia SPP. and thus have ascertained that when Salicornia SPP. is hydrolyzed and then purified, a salty sauce having improved sensory properties and antihypertensive activity (ACE inhibition activity) may be prepared without the additional use of a salt, whereby the active ingredient that exhibits antihypertension capability is 5-deoxy-irilin B (7-hydroxy-3-(2′-hydroxyphenyl)-6-methoxy-4H-1-benzopyran-4-one), which culminates in the present invention.

### DISCLOSURE

### Technical Problem

Accordingly, the present invention has been made keeping in mind the above problems encountered in the related art, and the present invention is intended to provide a Salicornia SPP.-derived salty sauce and a method of producing the same, wherein the salty sauce is derived from Salicornia SPP., and thus contains large amounts of nutrients such as minerals, amino acids and enzymes, is imparted with salty and savory tastes suitable for use as a seasoning sauce even without the addition of a salt, and may exhibit improved sensory properties and antihypertensive functionality.

In addition, the present invention is intended to provide a compound having superior ACE inhibition activity separated from Salicornia SPP., a method of separating the same, and a composition for the prevention and treatment of hypertension, containing it as an active ingredient.

### Technical Solution

Therefore, the present invention provides a Salicornia SPP.-derived salty sauce, containing a 5-deoxy-irilin B (7-hydroxy-3-(2′-hydroxyphenyl)-6-methoxy-4H-1-benzopyran-4-one) compound represented by Chemical Formula 1 below.

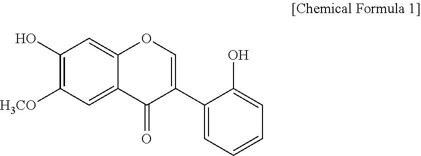 [SMILES: COc1cc2c(=O)c(-c3ccccc3O)coc2cc1O]

In the Salicornia SPP.-derived salty sauce according to the present invention, a sodium chloride (NaCl) content may be 6.0 to 39 wt % or a sodium content may be 2.4 to 15.8 wt %, and a weight ratio of potassium (K) to sodium (Na) may be 1:1 to 1:15.

In addition, the present invention provides a method of producing a Salicornia SPP.-derived salty sauce, comprising: (a) washing Salicornia SPP.; (b) cutting or grinding the washed Salicornia SPP.; (c) hydrolyzing the cut or ground Salicornia SPP., thus obtaining a Salicornia SPP. hydrolysate; (d) squeezing or extracting the Salicornia SPP. hydrolysate, thus obtaining a squeezed or extracted Salicornia SPP. liquid; (e) purifying the squeezed or extracted Salicornia SPP. liquid, thus obtaining a purified Salicornia SPP. liquid; and (f) concentrating the purified Salicornia SPP. liquid.

In addition, the present invention provides a method of producing a Salicornia SPP.-derived salty sauce, comprising: (a) washing Salicornia SPP.; (b) cutting or grinding the washed Salicornia SPP.; (c) squeezing or extracting the cut or ground Salicornia SPP., thus obtaining a squeezed or extracted Salicornia SPP. liquid; (d) purifying the squeezed or extracted Salicornia SPP. liquid, thus obtaining a purified Salicornia SPP. liquid; and (e) concentrating the purified Salicornia SPP. liquid.

The method of the invention may further comprise heating the cut or ground Salicornia SPP., after the cutting or grinding the washed Salicornia SPP.

In the present invention, the hydrolyzing may be performed using a biological process or a chemical process.

In the present invention, the purifying may be performed using at least one selected from the group consisting of a hydrophobic adsorbent, a cation exchange resin, and activated carbon.

In the present invention, the concentrating the purified Salicornia SPP. liquid may be performed so that the Salicornia SPP.-derived salty sauce has a solid content of 16 to 54 wt %.

In addition, the present invention provides a 5-deoxy-irilin B (7-hydroxy-3-(2′-hydroxyphenyl)-6-methoxy-4H-1-benzopyran-4-one) compound having angiotensin-I-converting enzyme (ACE) inhibition activity, represented by Chemical Formula 1.

In the present invention, the compound of Chemical Formula 1 may be separated from Salicornia SPP.

In addition, the present invention provides a composition for preventing and treating hypertension, containing, as an active ingredient, a 5-deoxy-irilin B (7-hydroxy-3-(2′-hydroxyphenyl)-6-methoxy-4H-1-benzopyran-4-one) compound, represented by Chemical Formula 1.

In addition, the present invention provides a method of separating a 5-deoxy-irilin B (7-hydroxy-3-(2′-hydroxyphenyl)-6-methoxy-4H-1-benzopyran-4-one) compound having angiotensin-I-converting enzyme (ACE) inhibition activity, the method comprising subjecting a Salicornia SPP. extract to organic solvent fractionation, column chromatography purification, and high-performance liquid chromatography (HPLC).

In the present invention, the Salicornia SPP. extract may be a methanol extract of a Salicornia SPP.-derived salty sauce obtained via hydrolysis using an enzyme.

### Advantageous Effects

According to the present invention, the Salicornia SPP.-derived salty sauce having superior sensory and functional properties can be produced (a) by cutting or grinding and then hydrolyzing Salicornia SPP. alone, without the addition of a saccharide source or supplementary materials, thus increasing the amounts of functional polyphenol and glutamic acid, which is responsible for a savory taste, (b) by purifying the hydrolysate, thus improving sensory properties such as removal of unpleasant tastes and odors and decoloration, and (c) by performing a concentration process under reduced pressure, thus exhibiting a savory taste and a salty taste suitable for use as a seasoning sauce even without the artificial addition of a salt.

Also, the Salicornia SPP.-derived salty sauce having sensory properties can be recovered through purification using a hydrophobic adsorbent, an ion exchange resin or activated carbon and concentration under reduced pressure, without the hydrolysis process.

According to the present invention, the Salicornia SPP.-derived salty sauce contains components of Salicornia SPP., for example, a salty component, such as sodium chloride contained therein, and an amino acid component such as glutamic acid and aspartic acid, which is responsible for a savory taste, and is thus useful in salty and savory dishes without the need to add other food additives.

### DESCRIPTION OF DRAWINGS

FIG. 1 shows a process of producing a Salicornia SPP.-derived salty sauce according to an embodiment of the present invention;

FIG. 2 is a graph showing the results of sensory testing of the Salicornia SPP.-derived salty sauce according to the present invention;

FIG. 3 is a graph showing the results of comparison of the ACE inhibition activity of Salicornia SPP.-derived salty sauces;

FIGS. 4A and 4B are graphs showing the results of inhibition of raised blood pressure due to salt intake in spontaneously hypertensive rats (SHRs) when using the enzyme-digested Salicornia sauce according to the present invention, FIG. 4A illustrating the systolic blood pressure and FIG. 4B illustrating diastolic blood pressure;

FIG. 5 shows chromatograms of high-performance liquid chromatography (HPLC) of the methanol extracts of Salicornia SPP.-derived salty sauces according to the present invention, in which the arrow-marked peaks indicate 5-deoxy irilin B;

FIGS. 6A to 6C show chromatograms of analytical and preparative HPLC of Compound A isolated from EDS-LH-7, corresponding to the fraction having ACE inhibition activity, purified through LH-20 column chromatography from the enzyme-digested Salicornia sauce according to the present invention, FIG. 6A illustrating the analytical HPLC chromatogram of EDS-LH-7, FIG. 6B illustrating the preparative HPLC chromatogram of EDS-LH-7, and FIG. 6C illustrating the HPLC chromatogram of isolated EDS-LH-7b (Compound A);

FIG. 7 is a graph showing the results of comparison of the ACE inhibition activity of main active fractions obtained during the purification of active ingredients having ACE inhibition activity from the enzyme-digested Salicornia sauce according to the present invention, in which EDS-M designates the methanol extract of salty sauce, EDS-MEA designates the ethyl acetate fraction of EDS-M, EDS-HP-3 designates the HP-20 column chromatography fraction of EDS-MEA, EDS-SC-3 designates the silica gel column chromatography fraction of PS-HP-3, EDS-LH-7 designates the LH-20 column chromatography fraction of EDS-SC-3, and EDS-LH-7a and EDS-LH-7b are compounds isolated from the EDS-LH-7 fraction;

FIG. 8 shows ESI-MS spectra obtained as the results of scanning Compound A, isolated according to the present invention, in positive and negative modes; and

FIGS. 9A to 9C show the results of nuclear magnetic resonance (NMR) of Compound A isolated according to the present invention, FIG. 9A illustrating the ¹H-NMR spectrum, FIG. 9B illustrating the HMBC-NMR spectrum, and FIG. 9C illustrating the structure of 5-deoxy-irilin B (7-hydroxy-3-(2′-hydroxyphenyl)-6-methoxy-4H-1-benzopyran-4-one) through ¹H-¹³C HMBC two-dimensional NMR spectroscopy.

### BEST MODE

The present invention has been undertaken to confirm the production of a Salicornia SPP.-derived salty sauce having superior sensory and functional properties when the hydrolysate, resulting from hydrolyzing cut or ground Salicornia SPP., is purified and concentrated.

In the present invention, Salicornia SPP. was cut and ground and then hydrolyzed with microorganisms, enzymes or acid/alkali. As the result thereof, the amounts of seasoning components such as free amino acids and the like and polyphenol and flavonoid functional components were confirmed to increase.

Also in the present invention, the hydrolysate was treated using a hydrophobic adsorbent or an ion exchange resin to thus increase the amounts of useful nutrients and functional components, and was purified using activated carbon. As the result thereof, it can be confirmed that the inherent bitter taste and unpleasant odor of Salicornia SPP. are removed and that it is decolored, thus improving sensory properties such as taste, flavor and color.

In the present invention, the purified Salicornia SPP. liquid was concentrated under reduced pressure. As the result thereof, it can be confirmed that the amounts of nutrients are further increased, and not only a savory taste but also a salty taste are sufficiently obtained, even without the artificial addition of a salt.

In the present invention, the salty sauce obtained after hydrolysis using microorganisms, enzymes or acid/alkali can be confirmed to exhibit remarkably high ACE inhibition activity and SHR-antihypertensive activity compared to those of the salty sauce before hydrolysis.

In the present invention, the ACE-inhibiting active ingredient was purified and isolated from the enzyme-digested Salicornia sauce having the greatest antihypertensive activity, and was then structurally analyzed and thus identified to be 5-deoxy-irilin B (7-hydroxy-3-(2′-hydroxyphenyl)-6-methoxy-4H-1-benzopyran-4-one).

Accordingly, the present invention addresses a Salicornia SPP.-derived salty sauce containing a 5-deoxy-irilin B (7-hydroxy-3-(2′-hydroxyphenyl)-6-methoxy-4H-1-benzopyran-4-one) compound represented by Chemical Formula 1 below.

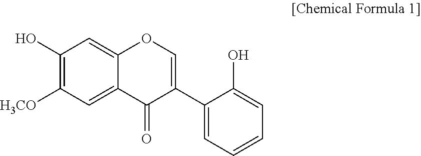 [SMILES: COc1cc2c(=O)c(-c3ccccc3O)coc2cc1O]

The Salicornia SPP.-derived salty sauce has a sodium chloride (NaCl) content of 6.0 to 39 wt % or a sodium content of 2.4 to 15.8 wt %, with a weight ratio of potassium (K) to sodium (Na) ranging from 1:1 to 1:15.

The Salicornia SPP.-derived salty sauce contains amino acid including glutamic acid. In particular, the glutamic acid content may fall in the range of 400 mg/100 g to 1,800 mg/100 g.

In the present invention, any Salicornia SPP. may be used without particular limitation, so long as it is typically known. For reference, Salicornia SPP. is an annual halophyte belonging to Chenopodiaceae. It grows in the foreshore or high-salt beach areas where typical plants have difficulty growing, and the habitat thereof is widely distributed over the world, including Korea, Europe and North America. Salicornia SPP. has a knobby stem, is fleshy and large, shows a dark green color, and is as high as 20 to 40 cm.

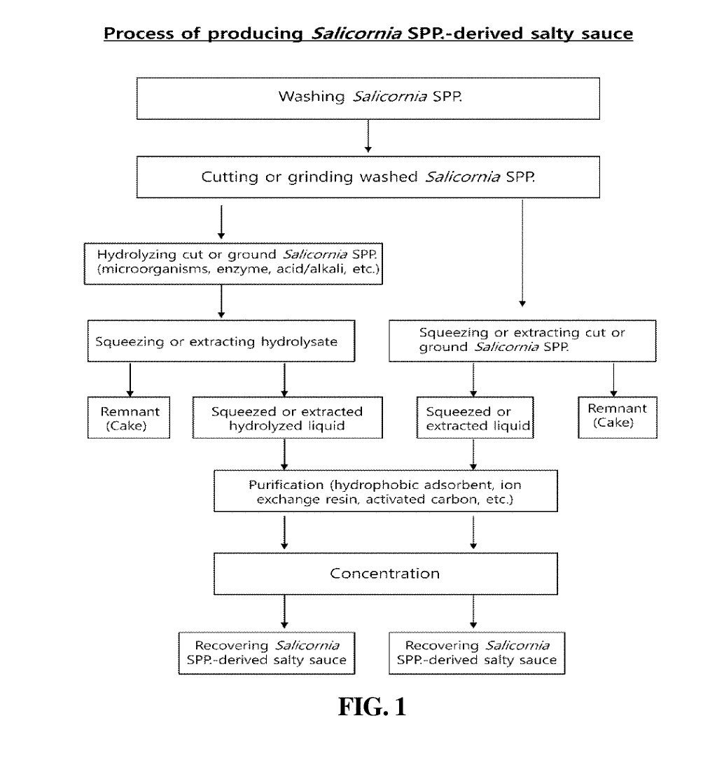

As shown in FIG. 1, the Salicornia SPP.-derived salty sauce according to the present invention may be produced in two manners.

In the first manner, the method of producing the Salicornia SPP.-derived salty sauce according to the present invention comprises the steps of (a) washing Salicornia SPP., (b) cutting or grinding the washed Salicornia SPP., (c) hydrolyzing the cut or ground Salicornia SPP., thus obtaining a Salicornia SPP. hydrolysate, (d) squeezing or extracting the Salicornia SPP. hydrolysate, thus obtaining a squeezed or extracted Salicornia SPP. liquid, (e) purifying the squeezed or extracted Salicornia SPP. liquid, thus obtaining a purified Salicornia SPP. liquid, and (f) concentrating the purified Salicornia SPP. liquid.

In the second manner, the hydrolysis step is omitted, and specifically, the method of producing the Salicornia SPP.-derived salty sauce according to the present invention comprises the steps of (a) washing Salicornia SPP., (b) cutting or grinding the washed Salicornia SPP., (c) squeezing or extracting the cut or ground Salicornia SPP., thus obtaining a squeezed or extracted Salicornia SPP. liquid, (d) purifying the squeezed or extracted Salicornia SPP. liquid, thus obtaining a purified Salicornia SPP. liquid, and (e) concentrating the purified Salicornia SPP. liquid.

In the step of washing Salicornia SPP., impurities such as soil and the like are removed from Salicornia SPP., and fresh and dry Salicornia SPP. may be used.

In the step of cutting or grinding the washed Salicornia SPP., the washed Salicornia SPP. may be finely cut or ground using a grinder.

In the present invention, the method of producing the Salicornia SPP.-derived salty sauce may further comprise heating the cut or ground Salicornia SPP. in order to increase functional and sensory properties, after the step of cutting or grinding the washed Salicornia SPP.

The step of hydrolyzing the cut or ground Salicornia SPP. may be performed using a biological process or a chemical process. The biological hydrolysis process may be conducted using microorganisms or enzymes. Here, any microorganisms may be used without limitation, so long as they secrete protease, cellulase, β-glucanase, or amylase, and the use of microorganisms suitable for use in food processing is preferable. Examples of the microorganisms may include Bacillus sp., Aspergillus sp., Lactobacillus sp., and Leuconostoc sp.

The hydrolysis of the ground Salicornia SPP. using microorganisms is preferably carried out at the optimal temperature for the microorganisms used for the hydrolysis. Since there may occur cases where the growth of microorganisms is inhibited or is impossible depending on the salt content of the ground Salicornia SPP., the salt content and pH of the ground Salicornia SPP. for hydrolysis preferably match the optimal growth conditions of the microorganisms.

As for hydrolysis of the ground Salicornia SPP. using an enzyme, any enzyme may be used without particular limitation, so long as it may hydrolyze the ground Salicornia SPP. The use of an enzyme for food processing is preferable, and examples thereof may include protease (proteolytic enzyme, endopeptidase, papain), amylase (alpha-amylase, gluco-amylase), cellulase, beta-glucanase, hemicellulase, and pectinase. Also, when an enzyme that reacts at high temperatures is used, external microbial contamination may be suppressed, which is more desirable.

The chemical hydrolysis process is implemented using an acid or alkali. The acid is exemplified by hydrochloric acid (HCl) and the alkali is exemplified by sodium hydroxide (NaOH), but the present invention is not limited thereto. Here, HCl or NaOH is used, such that a final concentration is 0.1 M to 5 M. For example, HCl may be added such that the final pH of the hydrolyzed liquid is 2, and NaOH may be added such that the final pH of the hydrolyzed liquid is 11. Hydrolysis using an acid or alkali may be performed for 1 to 5 hr. After the completion of hydrolysis, the hydrolysate including the acid or alkali is preferably neutralized to pH 7 using an acid or alkali solution.

In the case where biological hydrolysis using microorganisms or enzymes or chemical hydrolysis is performed, the nutritional and functional properties of the resulting Salicornia SPP.-derived seasoning sauce may be improved and the flavor thereof may be enhanced.

The step of squeezing or extracting the Salicornia SPP. hydrolysate or squeezing or extracting the cut or ground Salicornia SPP. is carried out to separate a solid and a liquid from each other. Here, the squeezing process is performed using a typically known squeezer, and the extraction process may be conducted using an inorganic solvent or an organic solvent, or through filtration or centrifugation.

In the case of water extraction, the amount of water that is added may be adjusted to the range of 0.5 to 5 L per kg of Salicornia SPP. The extraction process may be performed through pressure extraction (maximum 130° C.), non-pressure extraction (100° C.), low-temperature extraction (70 to 90° C.), or room-temperature extraction, and the extraction time may be adjusted within the range from 30 min to 6 hr depending on the type of extraction process.

The step of purifying the squeezed or extracted Salicornia SPP. liquid is performed using at least one selected from among a hydrophobic adsorbent, an ion exchange resin and activated carbon, and purification using a hydrophobic adsorbent or an ion exchange resin and then activated carbon is preferably carried out.

When the squeezed or extracted Salicornia SPP. liquid is purified through chromatography using a hydrophobic adsorbent or an ion exchange resin or is purified using activated carbon, the functional content of the Salicornia SPP.-derived salty sauce is increased, and unpleasant tastes and odors, as well as colors, may be removed.

Typically, the squeezed or extracted Salicornia SPP. liquid (undiluted solution) tastes bitter and is thus difficult to use as an edible sauce. Hence, in order to improve the sensory and functional properties of the Salicornia SPP.-derived salty sauce according to the present invention, purification is conducted through chromatography using a hydrophobic adsorbent or an ion exchange resin or is implemented using activated carbon, thus removing unpleasant tastes, unpleasant odors, and color. In the purification with activated carbon, the amount of activated carbon is preferably set within the range of 4 to 10 wt % of the soluble solid content of the squeezed or extracted Salicornia SPP. liquid. If the amount of activated carbon is less than 4 wt %, the effect of removing unpleasant tastes, unpleasant odors and color is insignificant. On the other hand, if the amount thereof exceeds 10 wt %, useful components may be removed.

In the case where a pretreatment process such as centrifugation, filter pressing or filtration is performed before the purification with activated carbon, the activated carbon purification efficiency may be increased.

The step of concentrating the purified Salicornia SPP. liquid is performed to increase a salty taste, a savory taste and nutrient content so that the purified Salicornia SPP. liquid may be used as a salty sauce. To this end, concentration under reduced pressure, vacuum concentration, thin-film concentration, evaporation, and freezing may be utilized without limitation.

Typically, a Salicornia SPP. extract (undiluted solution) has a low sodium chloride (NaCl) content of 2 to 4 wt %, and is thus unsuitable for use as a sauce. However, when such an extract is concentrated, the salty taste, savory taste and nutrient content thereof may be increased.

In the present invention, concentrating the purified Salicornia SPP. liquid is preferably performed such that the soluble solid content of the Salicornia SPP.-derived salty sauce falls in the range of 16 to 54 wt %. If the solid content is less than 16 wt %, the amounts of inherent components for functional and sensory properties are low, thus deteriorating functional and sensory properties. On the other hand, if the solid content exceeds 54 wt %, the viscosity of the sauce may increase.

In addition, the present invention addresses a 5-deoxy-irilin B (7-hydroxy-3-(2′-hydroxyphenyl)-6-methoxy-4H-1-benzopyran-4-one) compound having ACE inhibition activity, represented by Chemical Formula 1 below, and a composition for the prevention and treatment of hypertension, containing such a compound as an active ingredient.

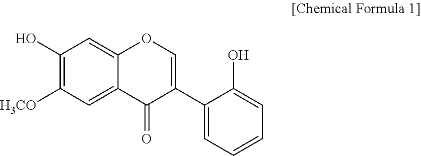 [SMILES: COc1cc2c(=O)c(-c3ccccc3O)coc2cc1O]

In the present invention, the 5-deoxy-irilin B (7-Hydroxy-3-(2′-hydroxyphenyl)-6-methoxy-4H-1-benzopyran-4-one) compound may be separated by subjecting the Salicornia SPP. extract to organic solvent fractionation, column chromatography purification, and HPLC.

Although not particularly limited, the Salicornia SPP. extract is a methanol extract of a Salicornia SPP.-derived salty sauce resulting from enzymatic hydrolysis.

According to the present invention, the composition for the prevention and treatment of hypertension may contain, in addition to 5-deoxy-irilin B (7-Hydroxy-3-(2′-hydroxyphenyl)-6-methoxy-4H-1-benzopyran-4-one), a pharmaceutically and physiologically acceptable assistant, and examples of the assistant may include a vehicle, a disintegrant, a sweetener, a binder, a coating agent, an expander, a lubricant, a glidant, and a flavoring agent.

The composition for the prevention and treatment of hypertension may be formulated into a pharmaceutical composition, containing at least one pharmaceutically acceptable carrier, in addition to the above active ingredient.

The pharmaceutical composition may be formulated in the form of a granule, a powder, a tablet, a coated tablet, a capsule, a suppository, a liquid, a syrup, a juice, a suspension, an emulsion, a drop or an injectable liquid. For example, in order to produce a tablet or capsule formulation, the active ingredient may be coupled with an oral non-toxic pharmaceutically acceptable inert carrier such as ethanol, glycerol, and water. If desired or required, an appropriate binder, lubricant, disintegrant or color former may be contained in combination therewith. The suitable binder may include, but is not limited to, natural saccharides, such as starch, gelatin, glucose or beta-lactose, natural and synthetic gums such as corn sweetener, acacia, Tragacanth or sodium oleate, sodium stearate, magnesium stearate, sodium benzoate, sodium acetate, and sodium chloride. The disintegrant may include, but is not limited to, starch, methyl cellulose, agar, bentonite, and xanthan gum. The pharmaceutically acceptable carrier in the composition formulated into a liquid solution may include at least one selected from among sterile and biocompatible saline, sterile water, Ringer's solution, buffer saline, albumin injection solution, dextrose solution, maltodextrin solution, glycerol, ethanol and mixtures. As necessary, any other additive such as an antioxidant, a buffer, a bacteriostatic agent or the like may be added. Also, a diluent, a dispersant, a surfactant, a binder and a lubricant may be additionally added, thus obtaining an injectable formulation such as an aqueous solution, a suspension, an emulsion, etc., or forming a formulation such as a pill, a capsule, a granule or a tablet.

Also, in the composition for the prevention and treatment of hypertension, an additional food or food component may be contained, in addition to the above active ingredient, and may be appropriately used through a typical method. The amount of the active ingredient that is mixed may be suitably determined depending on the end purpose (prevention, health or treatment). Generally, in the production of food or beverages, the composition of the present invention is added in an amount of 15 wt % or less, and preferably 10 wt % or less, based on the amount of raw material. However, in the case of long-term intake in order to improve health and hygiene or to maintain health, the amount thereof may be adjusted to be equal to or lower than the above range. Since there is no safety problem, the active ingredient may be used in an amount equal to or greater than the above range.

The kind of food is not particularly limited. Examples of the food to which the above material may be added may include sauce, meat, sausages, breads, chocolates, candies, snacks, confectionery, pizza, ramen, other noodles, gums, and dairy products such as ice cream, various soups, beverages, teas, drinks, alcoholic drinks, and vitamin complexes. Any typical health food is included.

In the case where the composition for the prevention and treatment of hypertension according to the present invention is a beverage composition, various flavoring agents or natural carbohydrates as in typical beverages may be additionally contained. The natural carbohydrate may include monosaccharide, such as glucose or fructose, disaccharide, such as maltose or sucrose, and a natural sweetener, such as dextrin or cyclodextrin, or a synthetic sweetener such as saccharin or aspartame. The amount of natural carbohydrate is about 0.01 to 10 g, and preferably about 0.01 to 0.1 g based on 100 mL of the composition of the present invention.

In addition thereto, the composition of the present invention may include any type of nutrient, vitamin, an electrolyte, a flavor, a colorant, pectic acid and salts thereof, alginic acid and salts thereof, organic acid, a protective colloidal thickener, a pH controller, a stabilizer, a preservative, glycerin, an alcohol, or a carbonating agent used in carbonated drinks. Also, the composition of the present invention may contain flesh of fruit for natural fruit juice, fruit juice drinks, and vegetable drinks. Such components may be used alone or in combination with one another. The amount of such an additive is not important, but falls in the range of 0.01 to 0.1 parts by weight based on 100 parts by weight of the composition of the present invention.

### MODE FOR INVENTION

A better understanding of the present invention may be obtained through the following examples which are set forth to illustrate, but are not to be construed as limiting the present invention.

The amounts of water, crude ash, carbohydrate, crude fat and crude protein of Salicornia SPP. were measured. The results are shown in Table 1 below.

TABLE 1

| Water | Crude ash | Carbohydrate | Crude fat | Crude protein |
|---|---|---|---|---|
| (%) | (%) | (%) | (%) | (%) |
| 78 | 6.2 | 12.8 | 0.26 | 2.8 |
| | (NaCl 4.54) | | | |

The composition and amounts (unit: mg/100 g) of amino acid for Salicornia SPP. were measured. The results are shown in Table 2 below.

TABLE 2

| | | | |
|---|---|---|---|
| Aspartic acid | 147.60 | Threonine | 74.0 |
| Serine | 78.3 | Alanine | 88.9 |
| Glutamic acid | 182.3 | Proline | 64.6 |
| Glycine | 93.4 | Cysteine | 3.7 |
| Histidine | 55.9 | Tyrosine | 3.6 |
| Tryptophan | 187.3 | Valine | 97.9 |
| Arginine | 56.7 | Methionine | 32.9 |
| Taurine | 22.2 | Lysine | 189.6 |
| Isoleucine | 104.3 | Leucine | 114.0 |
| Phenylalanine | 68.1 | Total | 1,665.3 |

The amounts (unit: mg/100 g) of minerals for Salicornia SPP. were measured. The results are shown in Table 3 below.

TABLE 3

| Fe | Zn | Ca | Na | Mg | K | Cu | Mn |
|---|---|---|---|---|---|---|---|
| 60.83 | 15.13 | 139.13 | 1,816.1 | 51.00 | 710.43 | 2.10 | 4.70 |

### Example 1: Cutting, Grinding and Hydrothermal Extraction of Salicornia SPP

1-1: Cutting and Grinding of Salicornia SPP.

Salicornia SPP., collected in September in the Sinan region, South Jeolla province, Korea, was washed two times with clean water to remove soil and impurities, and was then cut to a size of 2 to 10 mm using a cutter. The cut Salicornia SPP. was added with water in an amount 1.5 times the weight thereof, and ground for 5 min using a grinder with a cutting blade (rotations per minute of the rotating blade: 200 to 1,000), thus obtaining ground Salicornia SPP.

1-2: Hydrothermal Extraction of Salicornia SPP.

10 kg of the cut Salicornia SPP. was added with 15 L of water, and hydrothermally extracted at 105° C. for 5 hr using a high-speed low-temperature vacuum concentration extractor (made by Kyungseo E&P, COSMOS-660), thus obtaining a hydrothermally extracted liquid.

### Example 2: Hydrolysis of Ground Salicornia SPP

2-1: Hydrolysis Using Microorganisms

Bacillus sp. (Bacillus subtilis (KCTC No. 1021)) and Aspergillus sp. (Aspergillus niger (KCTC No. 6971) and Aspergillus oryzae (KCTC No. 6095)) were inoculated into a nutrient medium or agar and into a potato dextrose medium or agar, and incubated at 37° C. and 28° C. for 18 to 24 hr, and the broth thereof was added in an amount of 1 wt % based on the total weight of the ground Salicornia SPP. obtained in Example 1, followed by fermentation in a shaking thermostat (37° C.) for 24 to 120 hr, thus obtaining a Salicornia SPP. hydrolysate. The hydrolysate was squeezed using a squeezer (Hurom HVS-STF14), thus obtaining a hydrolyzed liquid. Also, the hydrolysate was primarily filtered with a filter cloth and then filtered using a filtration device having openings of 20 to 70 μm, thus yielding a hydrolyzed liquid.

2-2: Hydrolysis Using Enzyme

The ground Salicornia SPP. of Example 1 was treated with a protease (proteolytic enzyme, endopeptidase, papain) (made by Dupont, Vision Biochem), amylase (alpha-amylase, gluco-amylase) (made by Genencor), cellulase (made by AB Enzyme), beta-glucanase (made by AB Enzyme, Danisco), hemicellulase (made by AB Enzyme) and pectinase (made by Vision Biochem) and thus hydrolyzed. Each enzyme used in this Example was an enzyme having maximum activity in the pH range of 2.5 to 7.5. In particular, the enzyme having the maximum activity was used at a reaction temperature ranging from 40 to 70° C. The enzyme used for degrading the protein of Salicornia SPP. was a thermophilic enzyme having optimal conditions of a pH of 6.0 to 7.5 and a reaction temperature of 50 to 60° C., with an enzyme activity of 110,000 PC (Bacterial protease unit, the amount of enzyme for producing 1.5 μg/mL of L-tyrosine per minute)/g. Since the amount of protease that was added was 18.35% based on 100 g of the protein of Salicornia SPP., 1.66 g/100 mL of an enzyme was added. The α-amylase (Spezyme), which is an enzyme obtained from a Bacillus subtilis-derived broth, was purchased from Vision Biochem. The pH optimal for enzyme action was 6.2 to 7.0, and preferably 7.0, and the optimal temperature therefor was 70° C. The amount of the enzyme was added taking into consideration the amount of starch in the ground Salicornia SPP. The complex enzyme having the activity of cellulase, hemicellulase or beta-glucanase, that is, Rohament CL, was used, and the temperature and pH suitable for the action thereof were set to 60° C. and 5, respectively. The enzyme was added in consideration of the amounts of cellulose and glucan in the ground Salicornia SPP. as a substrate. The hydrolysate thus obtained was centrifuged (12,000 rpm, 20 min), thus obtaining a hydrolyzed liquid.

2-3: Hydrolysis Using Acid/Alkali

The ground Salicornia SPP. of Example 1-1 was added with HCl until the pH thereof was 2, hydrolyzed for 3 to 4 hr, and added with NaOH to neutralize it so that the pH thereof was 7, thus obtaining a hydrolysate.

Also, the ground Salicornia SPP. of Example 1-1 was added with NaOH until the pH thereof was 11, hydrolyzed for 3 to 4 hr, and added with HCl to neutralize it so that the pH thereof was 7, thus obtaining a hydrolysate. The hydrolysate thus obtained was squeezed using a squeezer (Hurom HVS-STF14), thus preparing a hydrolyzed liquid. Also, the hydrolysate was primarily filtered with a filter cloth and then filtered using a filtration device having openings of 20 to 70 μm, thus yielding a hydrolyzed liquid.

### Example 3: Purification

3-1: Purification Using Hydrophobic Adsorbent

In order to increase the functionality of the Salicornia SPP.-derived salty sauce and to remove unpleasant tastes and odors, the hydrolyzed liquid of Example 2-1 and the hydrothermally extracted liquid of Example 1-2 were purified through chromatography using porous hydrophobic adsorbents obtained by polymerizing styrene and divinylbenzene, respectively.

Specifically, a hydrophobic adsorbent was added to distilled water, allowed to stand at room temperature for about 12 hr to thus sufficiently supply water, placed in a column (30 mm (diameter)×400 mm (length)), and washed with water. Examples of the packing agent for purification were HP-20 (made by Mitsubishi Chemical) and SP-850 (made by Mitsubishi Chemical). 50% ethyl alcohol of 2 to 3 BV (Bed Volume) was passed at SV5 (Space Velocity) to replace the water present in resin voids and pores, and the column was allowed to stand for about 12 hr using a solution containing 50% ethyl alcohol, after which the packing agent and the alcohol in the column were replaced with distilled water. Into the upper portion of the column thus made, each of the hydrolyzed liquid of Example 2-1 and the hydrothermally extracted liquid of Example 1-2 was added in the same amount as the amount of the packing agent, and distilled water was passed therethrough at 2 to 3 BV, thus obtaining the purified Salicornia SPP. liquid.

3-2: Purification Using Ion Exchange Resin

In order to increase the functionality of the Salicornia SPP.-derived salty sauce, the hydrolyzed liquid of Example 2-2 was purified with an ion exchange resin (made by made by Mitsubishi Chemical, Japan). The cation exchange resin used for purification was DIAION WK60L (made by made by Mitsubishi Chemical, Japan) and the anion exchange resin used therefor was DIAION WA20 (made by Mitsubishi Chemical, Japan).

The cation exchange resin was reprocessed using 35% HCl, and the anion exchange resin was reprocessed using 95% NaOH. The concentration of the reprocessed solution was 1 mol. In the case of the cation exchange resin, 35% HCl was diluted about 1:11 by volume, and in the case of the anion exchange resin, 40 g of 95% NaOH was dissolved in 1 L of distilled water. The reprocessed ion exchange resin was treated to completely remove HCl or NaOH from the ion exchange resin using distilled water, and was then packed in the column. The column for purification using the ion exchange resin was made of glass having a size of 30 mm (inner diameter) and 400 mm (length). The prepared column was filled with the reprocessed and water-washed weak acid cation exchange resin and weak base anion exchange resin, and then washed with distilled water. The upper portion of the column thus made was added with the hydrolyzed liquid in the same amount as in the ion exchange resin, after which distilled water in an amount of 3 to 5 BV relative to the amount of the ion exchange resin was passed therethrough, thus obtaining the purified Salicornia SPP. liquid.

3-3: Purification Using Activated Carbon

In order to increase the sensory properties of the Salicornia SPP.-derived salty sauce by removing unpleasant tastes and odors therefrom, each of the hydrolyzed liquid of Example 2-1 and the hydrothermally extracted liquid of Example 1-2 was purified using activated carbon (powdered activated carbon having a particle size of 1 to 150 μm, Noritz Co., USA). The activated carbon was added in an amount of 4 to 8 wt % of the solid content of the hydrolyzed liquid or the hydrothermally extracted liquid, followed by purification at 80° C. for 30 min. After the completion of purification, the activated carbon was completely removed using a filtration process or a filter press process.

The Salicornia SPP. extract has an inherent characteristic unpleasant taste and unpleasant odor and is thus unsuitable for sensory edible purposes, but the taste, smell and color thereof were able to be improved through purification using activated carbon.

### Example 4: Concentration

The hydrophobic adsorbent-purified liquid of the hydrolyzed liquid, obtained in Example 3-1, was concentrated using a reduced-pressure concentrator with a bathtub at 40 to 60° C. so that the solid content of the concentrated liquid was 30 to 40 wt %, thus yielding a Salicornia SPP.-derived salty sauce.

### Test Example 1: Sensory Evaluation of Salicornia SPP.-Derived Salty Sauce

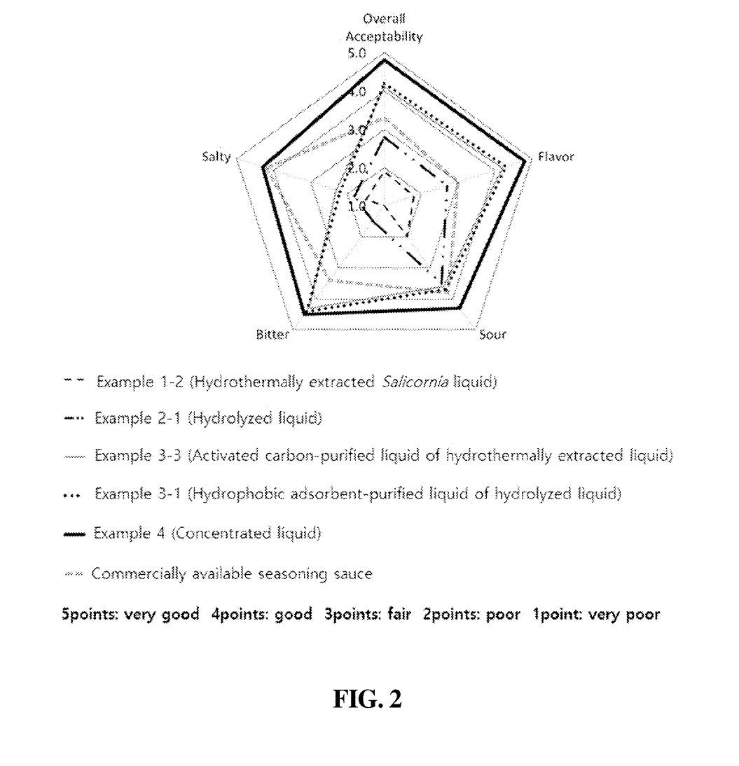

In order to evaluate sensory properties of the Salicornia SPP.-derived salty sauce prepared in the Example, sensory properties and color using a colorimeter were measured. The results are shown in Tables 4 and 5 below and FIG. 2.

Color was analyzed using a Hunter color difference meter (Super color sp-80 colorimeter, Tokyo Denshoku Co., Japan), with which the brightness (white 100 to 0 black), redness (red 100 to −80 green), and yellowness (yellow 70 to −80 black) were measured. The color coordinates of standard plate were L of 94.70, a of −0.61, and b of 4.04, and three measurements were performed per sample. The average values thereof are shown in Table 4 below.

The sensory properties were evaluated on a 5-point scale by 20 researchers, 1 point indicating very poor, 2 points indicating poor, 3 points indicating fair, 4 points indicating good, and 5 points indicating very good.

TABLE 4

| Variety | L | A | b |
|---|---|---|---|
| Ex. 1-2 (Hydrothermally | 32.20 ± 0.09 | 10.45 ± 0.12 | 14.19 ± 0.16 |
| extracted Salicornia | | | |
| liquid) | | | |
| Ex. 2-1 (Microbiologically | 31.98 ± 0.02 | 10.55 ± 0.02 | 12.41 ± 0.01 |
| hydrolyzed liquid) | | | |
| Ex. 3-1 (Hydrophobic | 42.88 ± 0.13 | 7.60 ± 0.16 | 16.30 ± 0.49 |
| adsorbent-purified | | | |
| liquid of hydrolyzed | | | |
| liquid) | | | |
| Ex. 3-3 (Activated carbon- | 41.94 ± 0.41 | 5.30 ± 0.04 | 18.15 ± 0.32 |
| purified liquid of | | | |
| hydrothermally | | | |
| extracted liquid) | | | |
| Ex. 4 (Concentrated liquid) | 36.34 ± 0.55 | 4.08 ± 0.17 | 21.15 ± 0.17 |
| Commercially available | 34.06 ± 0.30 | 4.48 ± 0.21 | 18.85 ± 0.16 |
| seasoning sauce | | | |

TABLE 5

| | | Sensor Evaluation | | | | |
|---|---|---|---|---|---|---|
| | | Overall | | | | |
| Variety | Procedure | Acceptability | Flavor | Sour | Bitter | Salty |
| Salicornia | Ex. 1-2 | 1.9 ± 0.4 | 1.8 ± 0.7 | 2.0 ± 0.4 | 1 ± 0.1 | 1.5 ± 0.1 |
| extract | (Hydrothermally | | | | | |
| | extracted | | | | | |
| | Salicornia liquid) | | | | | |
| | Ex. 2-1 | 2.8 ± 0.2 | 2.7 ± 0.4 | 3.5 ± 0.4 | 1.5 ± 0.6 | 1.8 ± 0.5 |
| | (Microbiologically | | | | | |
| | hydrolyzed liquid) | | | | | |
| | Ex. 3-1 | 4.2 ± 0.3 | 4.3 ± 0.1 | 3.7 ± 0.1 | 4.4 ± 0.4 | 2.2 ± 0.4 |
| | (Hydrophobic | | | | | |
| | adsorbent-purified | | | | | |
| | liquid of | | | | | |
| | hydrolyzed liquid) | | | | | |
| | Ex. 3-3 (Activated | 4.1 ± 0.1 | 4.2 ± 0.5 | 3.6 ± 0.4 | 4.3 ± 0.5 | 2.3 ± 0.1 |
| | carbon-purified | | | | | |
| | liquid of | | | | | |
| | hydrothermally | | | | | |
| | extracted liquid) | | | | | |
| | Ex. 4 (Concentrated | 4.8 ± 0.2 | 4.8 ± 0.3 | 4.3 ± 0.3 | 4.5 ± 0.1 | 4.3 ± 0.1 |
| | liquid) | | | | | |

| | | | | | |
|---|---|---|---|---|---|
| Commercially available | 3.3 ± 0.1 | 3.0 ± 0.2 | 3.8 ± 0.1 | 3.4 ± 0.6 | 4.2 ± 0.1 |
| seasoning sauce | | | | | |

As is apparent from Tables 4 and 5 and FIG. 2, the hydrothermally extracted Salicornia liquid of Example 1-2 had excessively low salt content, a very bitter taste, and a dark color, and was thus unsuitable for use as a sauce.

When comparing the sensory properties of the microbiologically hydrolyzed liquid of Example 2-1 and the hydrophobic adsorbent-purified liquid of the hydrolyzed liquid of Example 3-1, the bitter taste and unpleasant odor of Salicornia SPP. can be confirmed to be removed through purification using a hydrophobic adsorbent.

When comparing the sensory properties of the hydrothermally extracted Salicornia liquid of Example 1-2 and the activated carbon-purified liquid of the hydrothermally extracted liquid of Example 3-3, the bitter taste and unpleasant odor of Salicornia SPP. can be confirmed to be removed through purification using activated carbon.

When comparing the sensory properties of the concentrated Salicornia SPP.-derived salty sauce of Example 4 and the commercially available seasoning sauce (namely, a liquid sauce marketed under the trade name “Reason why cooking is delicious”, made by Sinsong Food), the Salicornia SPP.-derived salty sauce of the present invention can be confirmed to have a salty taste sufficient for use as a salty sauce and superior taste and flavor compared to the commercially available seasoning sauce to thus exhibit high overall acceptability.

### Test Example 2: Evaluation of Component of Salicornia SPP.-Derived Salty Sauce

In order to evaluate the availability of the Salicornia SPP.-derived salty sauce of Example 4, component analysis for NaCl, minerals, water and amino acids was performed. The results are shown in Tables 6 and 7 below.

TABLE 6

| Specification | Salicornia SPP.-derived salty sauce |
|---|---|
| Sodium Chloride (NaCl) | 10 to 24% (w/v) |

| | | |
|---|---|---|
| Minerals | K | 1,567 to 2,340 mg/100 g |
| | Mg | 150 to 201 mg/100 g |
| | Ca | 380 to 495 mg/100 g |

| | |
|---|---|
| Water Content | 67 to 70% (w/v) |

As is apparent from Table 6, the Salicornia SPP.-derived salty sauce of the present invention had a NaCl content of 10 to 24 wt % and was thus suitable for use as a salty sauce.

Also, it had K content of 1,567 to 2,340 mg/100 g, Mg content of 150 to 201 mg/100 g, and Ca content of 380 to 495 mg/100 g, and thus the mineral content of the salty sauce of the invention was remarkably increased compared to that of typical Salicornia SPP. of Table 3.

TABLE 7

| | | | |
|---|---|---|---|
| Aspartic acid | 653.5 | Threonine | 297.8 |
| Serine | 259.5 | Alanine | 402.3 |
| Glutamic acid | 1,587.6 | Proline | 246.2 |
| Glycine | 312.2 | Cysteine | 10.9 |
| Histidine | 143.2 | Tyrosine | 11.2 |
| Arginine | 213.1 | Valine | 367.9 |
| Phenylalanine | 189.6 | Methionine | 126.2 |
| Leucine | 309.8 | Lysine | 681.2 |
| Isoleucine | 346.1 | Total | 7,017.0 |

As is apparent from Table 7, in the Salicornia SPP.-derived salty sauce of the present invention, the amounts (unit: mg/100 g) of amino acids including glutamic acid and the like were remarkably increased compared to typical Salicornia SPP. of Table 2.

### Test Example 3: Evaluation of Functionality of Salicornia SPP.-Derived Salty Sauce

3-1: Evaluation of Amounts of Functional Polyphenol and Flavonoid

In order to evaluate the amounts of functional components of the hydrolyzed Salicornia salty sauces of Example 2 and the non-hydrolyzed salty sauce of Example 1, the amounts of polyphenol and flavonoid compounds were measured. The results are shown in Table 8 below.

The total polyphenol content was measured on a 96-well microplate using a modified Folin-Davis method. The non-hydrolyzed salty sauce of Example 1 and the hydrolyzed Salicornia salty sauces of Example 2 were extracted with 70% methanol and then dried, after which the resulting samples were dissolved in distilled water at various concentrations to give 20 μL of each sample liquid, which was then mixed with 250 μL of 2% sodium carbonate, added with 15 μL of a 50% Folin-Ciocalteu (Sigma Co., USA) solution, allowed to stand at room temperature for 30 min and measured for absorbance at 725 nm using a Microreader (Bio-RAD, x-Mark, USA). As a standard reagent, 0 to 500 μg/mL of a tannic acid (Sigma Co., USA) solution was reacted in place of the sample to obtain a calibration curve, from which the total polyphenol content contained in the extracted sample was then calculated.

The total flavonoid content was measured on a 96-well microplate using a modified Abdel-Hameed method. The non-hydrolyzed salty sauce of Example 1 and the hydrolyzed Salicornia salty sauces of Example 2 were extracted with 70% methanol and dried, after which the resulting samples were dissolved in distilled water at various concentrations to give 30 μL of each sample liquid, which was then added with 200 μL of 90% diethylene glycol, further added with 5 μL of 1 N NaOH, reacted at 37° C. for 1 hr, and measured for absorbance at 420 nm using a Microreader (Bio-RAD, x-Mark, USA). As a standard reagent, 0 to 500 μg/mL of rutin (Sigma Co., USA) was reacted in place of the sample to obtain a calibration curve, from which the total flavonoid content contained in the extracted sample was then calculated.

TABLE 8

| | mg/100 g | |
|---|---|---|
| Salicornia salty sauces before and | Total | Total |
| after hydrolysis | Polyphenol | Flavonoid |
| Hydrothermally extracted salty sauce | 1980.4 | 489.7 |
| (Ex. 1-2) | | |
| Microbiologically hydrolyzed salty | 2872.0 | 897.6 |
| sauce (Ex. 2-1) | | |
| Enzyme-digested salty sauce (Ex. 2-2) | 3591.2 | 1276.8 |
| Acid/alkali-hydrolyzed salty sauce | 2354.9 | 685.1 |
| (Ex. 2-3) | | |

As is apparent from Table 8, the total polyphenol content and the total flavonoid content, which are main functional components in the hydrolyzed Salicornia salty sauces, were considerably increased compared to those of the non-hydrolyzed salty sauce. In particular, the enzyme-digested salty sauce had very high functional polyphenol and flavonoid contents compared to those of the microbiologically hydrolyzed and acid/alkali-hydrolyzed salty sauces.

3-2: Evaluation of ACE Inhibition Activity

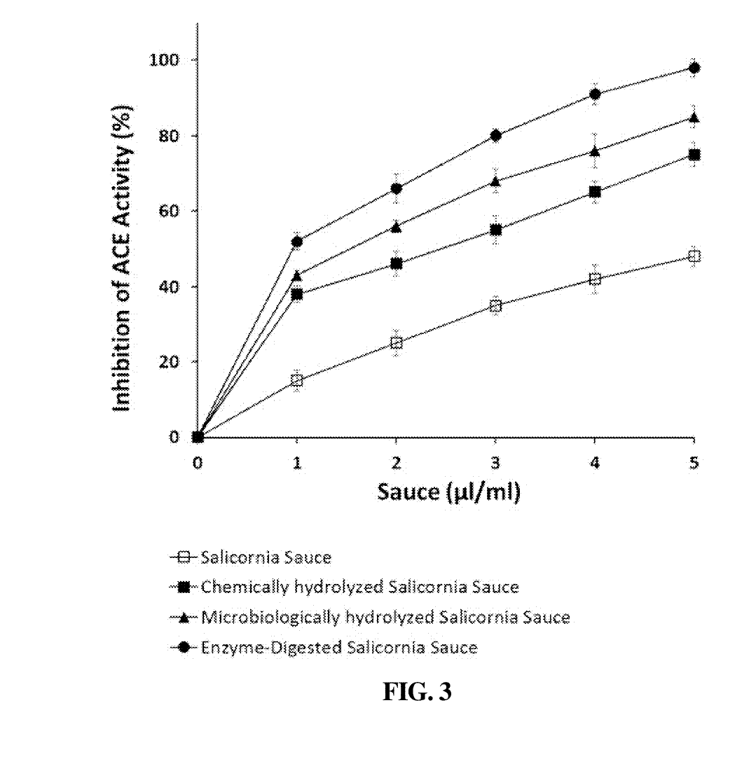

As shown in the results of Table 8, the hydrolyzed Salicornia salty sauces had very high functional polyphenol and flavonoid contents compared to the non-hydrolyzed salty sauce. Thus, as an indicator for antihypertensive activity, angiotensin-I-converting enzyme (ACE) inhibition activity was evaluated. As the test sample, each salty sauce, adjusted to a salt content of 14%, was added at a concentration of 1 to 5 μL/mL to the reaction solution, and then measured. The results are shown in FIG. 3.

Angiotensin-II, produced by ACE, promotes an increase in blood pressure and secretion of antidiuretic hormone aldosterone, and suppresses the emission of water and sodium to thus increase blood circulation, thus causing hypertension. Hence, to evaluate the ACE inhibition activity, ACE (Angiotensin I-Converting Enzyme) inhibition activity was measured as follows. Specifically, 1 g of a rabbit lung acetone powder (Sigma Col) in 50 μL of the sample was mixed with 25 μL (2.5 unit) of an ACE supernatant dissolved in a 0.1 M sodium borate buffer containing 10 mL of 0.3 M NaCl, 50 μL of a 0.1 M sodium borate buffer (pH 8.3) containing 0.3 M NaCl, and 25 μL of a sample solution at various concentrations (0.25, 0.5 and 1.0 mg/mL), and then preincubated at 37° C. for 10 min. Further, 50 μL of a Hip-His-Leu substrate solution was added, and the resulting mixture was allowed to react at 37° C. for 30 min, after which the reaction was stopped by the addition of 100 μL of 1 N HCl. 1 mL of ethyl acetate was added, and the resulting reaction mixture was agitated in a vortex for 1 min and centrifuged at 3,000 g for 15 min, after which 0.8 mL of the separated ethyl acetate supernatant (extract) was recovered. The supernatant was warmed in a hood and thus completely volatilized, and was then dissolved in 1 mL of a sodium borate buffer under the same conditions, after which absorbance at 228 nm was measured and ACE inhibition activity was calculated.

As shown in FIG. 3, the Salicornia salty sauce before hydrolysis at a concentration of 1 μL/mL exhibited ACE inhibition activity of about 16.5%, but the ACE inhibition activity of the salty sauces after hydrolysis using microorganisms, enzymes and chemical acid/alkali was approximately doubled. The hydrolyzed Salicornia salty sauce of the present invention can be found to have significantly improved antihypertensive functionality, which is deemed to be associated with the increased amounts of functional polyphenol and flavonoid in the hydrolyzed salty sauce, as is apparent from Table 7.

3-3: Evaluation of Antihypertensive Effect in SHR

The enzyme-digested salty sauce was confirmed to exhibit the greatest ACE inhibition activity, as shown in FIG. 3, and the ACE inhibition activity of the hydrothermally extracted Salicornia salty sauce of Example 1-2 and the enzyme-digested Salicornia salty sauce of Example 2-2 was evaluated using a blank group and a control group (SHR-NaCl Solution), in which an NaCl solution was administered at the same concentration as in the salty sauce. In the salty sauce-administered test group, 6-week-old SHRs (Spontaneously Hypertensive Rats) were administered repetitively for 8 weeks with the hydrothermally extracted Salicornia salty sauce of Example 1-2 and the enzyme-digested Salicornia salty sauce of Example 2-2, and the systolic blood pressure and diastolic blood pressure were measured and compared.

SHRs were purchased from Orient Bio, and had an average weight of 200 g±20%. SHRs were bred at a temperature of 20° C., a relative humidity of 50 to 60%, and a light-dark cycle of 12 hr, and water and feed (made by Cargill Agri Purina) were freely supplied. SHRs were adapted to the test conditions for 7 days and then weighed and thus classified into individual test groups. 10 rats were allocated to each test group.

Preparation of Test Material to be Administered:

The test materials of the NaCl solution-administered group and the Salicornia salty sauce-administered group had NaCl in the same content, namely 14 wt %. The test material of the NaCl solution-administered group was prepared by dissolving an appropriate amount of NaCl in a vehicle. The vehicle was sterile saline (made by Dai Han Pharm.).

Setting of Dose:

When 4% NaCl-containing feed was supplied to rats under the condition that the average daily feed intake of a rat was about 20 g, rats were able to take about 800 mg of NaCl per day, and thus 800 mg of NaCl was set to be administered daily to the NaCl solution-administered group and the Salicornia salty sauce-administered group.

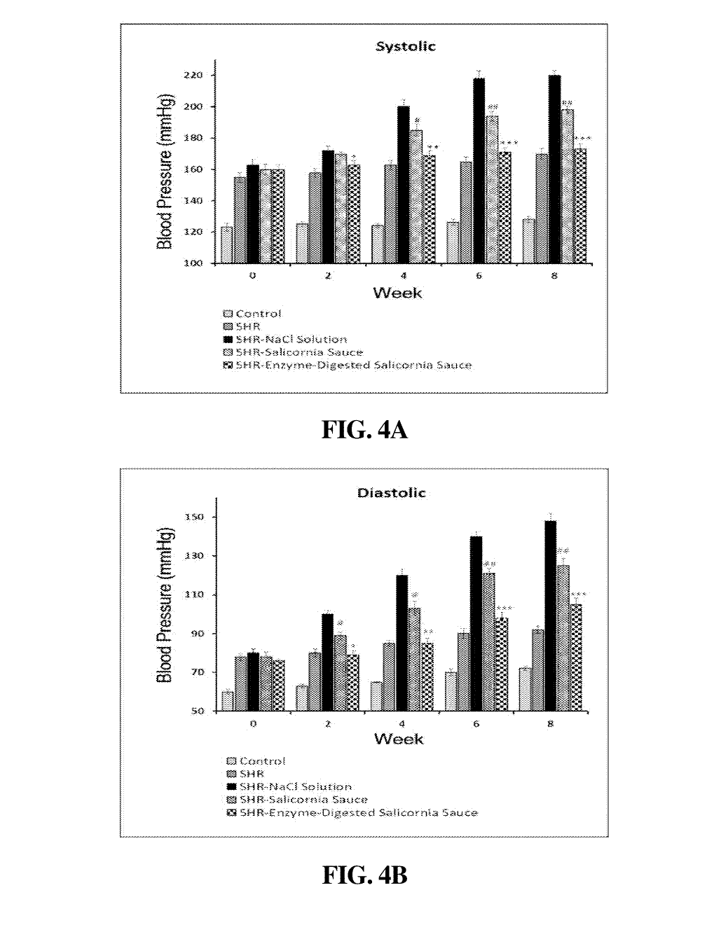

In order to evaluate the antihypertensive effect of the Salicornia Spp.-derived salty sauce, the systolic blood pressure and the diastolic blood pressure of the rats were measured with tail-cuff plethysmography (BP-2000; Visitech System, Apex, N.C., USA) at 2, 4, 6, and 8 weeks after the initiation of administration. The results are shown in FIGS. 4A and 4B.

As shown in FIGS. 4A and 4B, the systolic blood pressure and the diastolic blood pressure were continuously increased in the NaCl solution-administered group (SHR-NaCl Solution), whereas an increase in blood pressure was inhibited in the Salicornia salty sauce-administered group (SHR-Salicornia Sauce) compared to the NaCl solution-administered group. Particularly in the SHR-Enzyme-Digested Salicornia sauce, the antihypertensive effect was very significant compared to the non-hydrolyzed Salicornia salty sauce. This means that the Salicornia SPP.-derived salty sauce does not cause hypertension and also contains increased amounts not only of the component having ACE inhibition activity but also of the functional component for inhibiting an increase in blood pressure.

### Example 5: Isolation of Active Ingredient Having ACE Inhibition Activity from Salicornia Salty Sauce and Identification of Structure Thereof

5-1: HPLC of Methanol Extract of Salicornia Salty Sauce

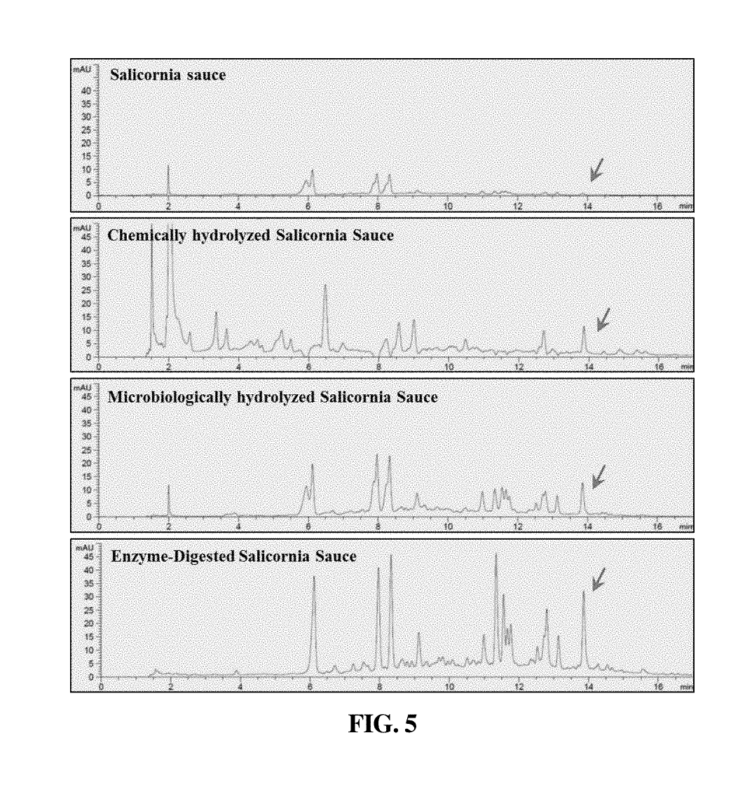

As is apparent from the results of Table 8, in order to more specifically analyze the profiles of active ingredients, 100 mg of a dry sample, obtained by lyophilizing each of the hydrothermally extracted salty sauce, the microbiologically hydrolyzed salty sauce, the enzyme-digested salty sauce and the chemically acid/alkali-hydrolyzed salty sauce, was added with 2 mL of methanol, extracted for 10 min using ultrasonic waves, and filtered to obtain a methanol extract, which was then filtered with a 0.22 μM ceiling filter for an organic solvent and subjected to HPLC (at 300 nm/390 nm, corresponding to the common UV absorption bands of functional polyphenols and flavonoids). The results are shown in FIG. 5.

As shown in FIG. 5, in the hydrolyzed Salicornia salty sauces compared to the hydrothermally extracted salty sauce, there was a greater variety of kinds of analytical components, and the concentrations thereof were relatively high. Similar to the quantitative evaluation of polyphenols and flavonoids given in Table 8, the greatest variety of kinds of materials were contained at high concentrations in the enzyme-digested salty sauce. The peak component appearing at a retention time of 13.9 min was identified to be a 5-deoxy irilin B compound, which was revealed as the ACE inhibition component in the present invention. Thereby, the 5-deoxy-irilin B compound was found to be contained at the highest concentration in the enzyme-digested Salicornia salty sauce having the greatest antihypertensive activity.

5-2: Purification of Active Ingredient Having ACE Inhibition Activity from Enzyme-Digested Salty Sauce

The methanol extract (50 g) of the enzyme-digested salty sauce was dissolved in 1.5 L of distilled water, mixed with 1.5 L of n-hexane in a 3 L separatory funnel and fractionated two times into the hexane layer and the water layer. Also, the water layer was added with 1.5 L of chloroform and fractionated two times into the chloroform layer and the water layer. Also, the water layer was added with ethyl acetate and fractionated two times into the ethyl acetate layer and the water layer. Finally, the water layer was added with 1.5 L of n-butanol and fractionated two times into the butanol layer and the water layer. Each solvent extract thus obtained was dried under reduced pressure to remove the corresponding organic solvent, and was then lyophilized, thus obtaining a hexane fraction (2.8 g), a chloroform fraction (1.8 g), an ethyl acetate fraction (5.4 g), a butanol fraction (8.6 g), and a water fraction (28.5 g). Among the organic solvent fractions, the ethyl acetate fraction (EDS-MEA) (5.4 g) which had the greatest ACE inhibition activity was dissolved in 100 mL of distilled water (pH 5), adsorbed to the upper portion of the first column (3×40 cm), which was packed with adsorptive DIAION HP-20 resin, and then sequentially eluted with distilled water, 30% methanol, 70% methanol and 70% acetone, thus obtaining four fractions. Among them, the 70% methanol elution fraction (EDS-HP-3, 2.2 g) having the greatest ACE inhibition activity was placed in the second column (3.3×40 cm), which was packed with polar silica gel, and was then eluted at a flow rate of 0.5 mL/min using a mobile phase comprising a mixture of methanol and chloroform at 1:1, thus obtaining five fractions in 300 mL each. Among them, the fraction (EDS-SC-3, 552 mg) having the greatest ACE inhibition activity was dried under reduced pressure, dissolved in 3 mL of methanol, placed in the third column (2.5×33 cm), which was packed with gel filtration Sephadex LH-20, and then eluted with 100% methanol (a flow rate of 0.2 mL/min) as a mobile phase solvent, thus obtaining a total of 18 fractions (EDS-LH-1 to EDS-L-18) in 40 mL each.

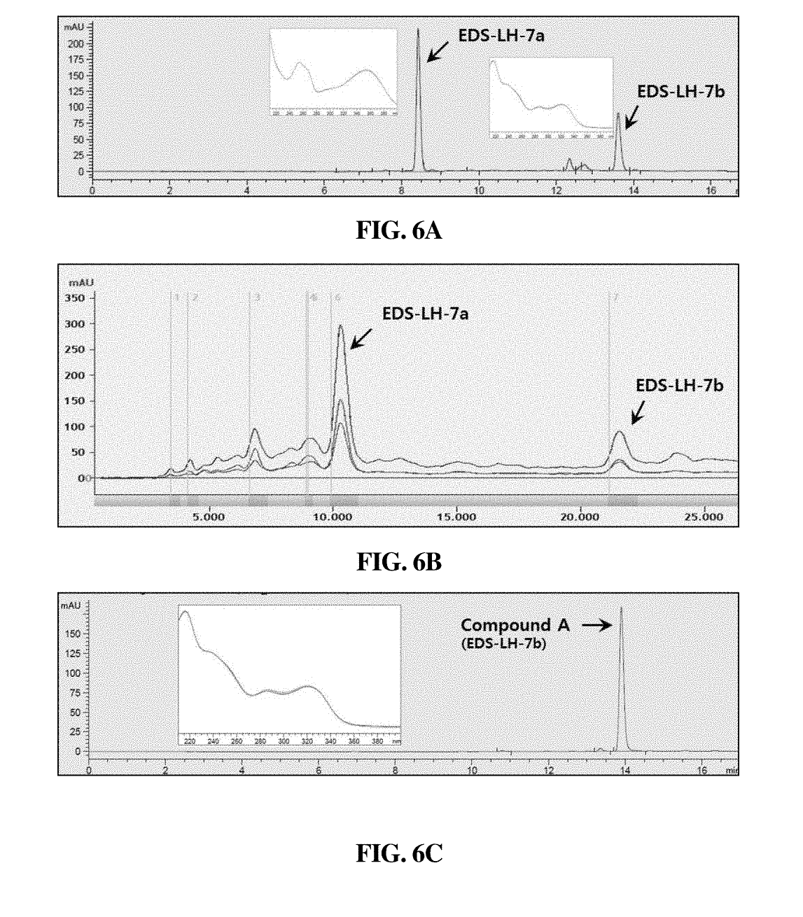

Finally, among the 18 fractions, the EDS-LH-7, having the greatest ACE inhibition activity, was concentrated under reduced pressure and lyophilized, and thus collected in an amount of 83 mg. mg of EDS-LH-7 was dissolved in 1 mL of methanol for HPLC, filtered with a 0.22 μm filter, and then subjected to HPLC. As the result thereof, two main peaks (EDS-LH-7a, EDS-LH-7b) were identified and isolated using prep-HPLC (FIGS. 6A to 6C). For analytical HPLC, a model (1260 Infinity, Agilent, USA) equipped with a Zorbax Eclipse C18 column (5 μm, 4.5×250 mm, Agilent) was used, and HPLC was performed using a Multiple Preparative HPLC (LC-forte/R, YMC, Japan) equipped with a preparative column (Triart C18, 20 mm×150 mm, 5 μm, YMC, Japan). As the mobile phase solvent, methanol and tertiary distilled water were allowed to flow at a rate of 1 to 3 mL/min under gradient conditions, and an Agilent 1200 DAD detector or YMC UV-3400 UV detector was used. The compounds were fractionated based on the absorbance in the two wavelength ranges (260 and 330 nm), thus obtaining EDS-LH-7a (35.4 mg) at a retention time of 10.5 min and EDS-LH-7b (18.2 mg) at a retention time of 21.5 min (FIGS. 6A to 6C).

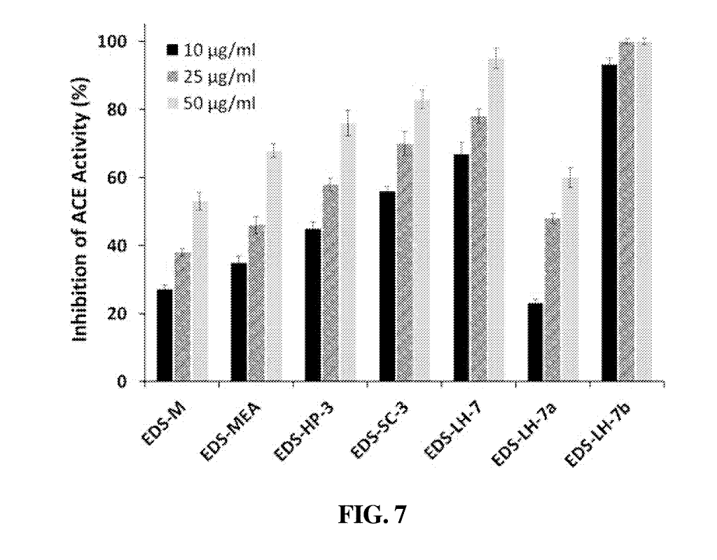

During purification, the organic solvent fractions (at concentrations of 10, 25, 50 μg/mL) were measured for ACE inhibition activity in the same manner as in Example 3-2. The results are shown in FIG. 7.

As shown in FIG. 7, as the purification progressed, the ACE inhibition activity of the fractions was continually increased. Finally, the ACE inhibition activity of EDS-LH-7b (Compound A), isolated from EDS-LH-7, was the greatest, reaching a level of 95% or more at a concentration of 10 μg/mL, whereby this compound was confirmed to be the antihypertensive active ingredient of the Salicornia SPP.-derived salty sauce.

Also, IC₅₀ values of EDS-LH-7b (Compound A) and Luteolin, known to have very high ACE inhibition activity among plant flavonoid components, were measured. The results are shown in Table 9 below.

TABLE 9

| Compound | ACE inhibition activity IC₅₀ (μg/mL) |
|---|---|
| EDS-LH-7b (Compound A) | 4.5 |
| Luteolin | 26.7 |

As is apparent from Table 9, IC₅₀ (4.5 μg/mL) of EDS-LH-7b, showing 50% ACE inhibition activity, was at least five times superior to IC₅₀ (26.7 μg/mL) of Luteolin.

5-3: Analysis of Structure of Compound a from Salicornia SPP.-Derived Salty Sauce

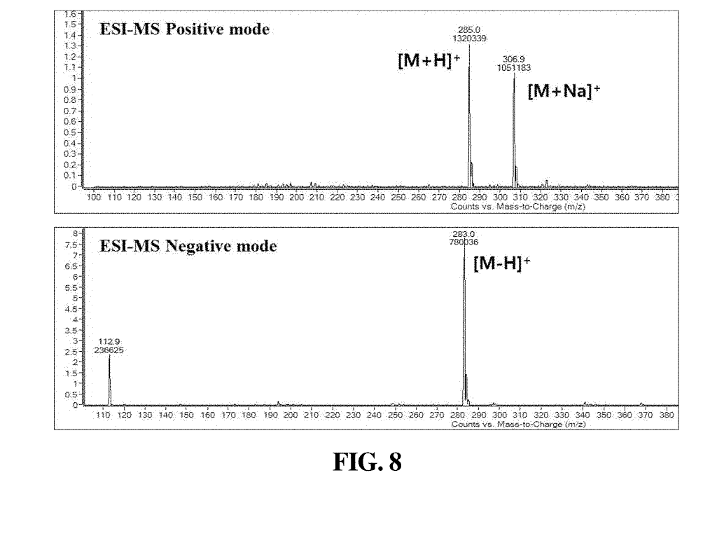

The maximum UV absorption range of Compound A (EDS-LH-7b), isolated in Example 5-2, and the adsorption shift due to the addition of a reagent were measured in the range of 190 to 500 nm using a UV spectrophotometer (Genesys 10S UV-VIS spectrophotometer, Thermo Scientific, USA) by dissolving each sample at a concentration of 1 mg/mL in methanol. In order to observe changes in the UV absorption range due to the addition of the reagent, AlCl₃, NaOH and NaOAc were used. To determine the molecular weight of Compound A, 1 mg of Compound A was subjected to positive and negative scanning using an electrospray ionization (ESI) mass spectrometer (LC-ESI mass spectrometer, AGILENT 1100, USA Micromass Quattro II), and high-resolution MS was measured. The results are shown in FIG. 8.

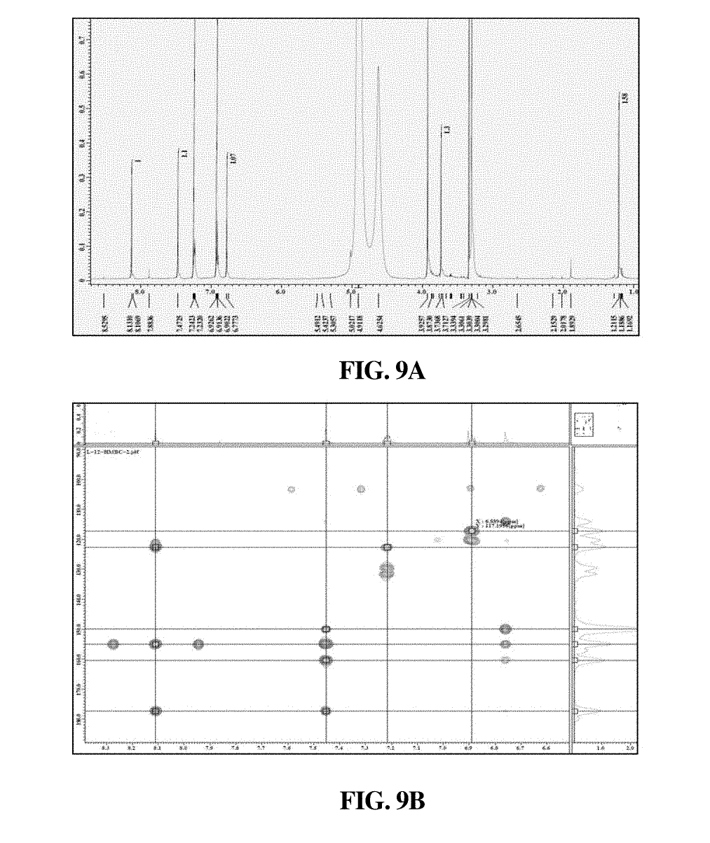

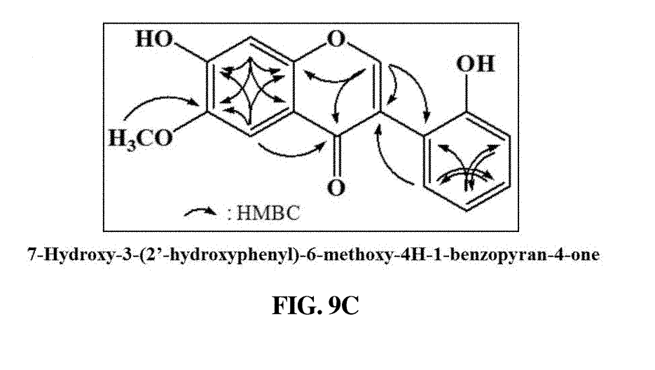

NMR spectroscopy was performed in a manner in which Compound A (3 mg) was completely dried, dissolved in DMSO d6 (0.5 mL), placed in a 5 mm NMR tube, and analyzed using a JNM-ECA 600, Jeol, Japan, and ¹H-NMR (FIG. 9A) and ¹³C-NMR were measured at 600 MHz and at 150 MHz, respectively. Through ¹H-¹H COSY, HMQC, and HMBC spectrum measurement, the positions of hydrogen and carbon in Compound A were determined (FIG. 9B).

Based on the measurement results, Compound A was identified to be 5-deoxy-irilin B (7-hydroxy-3-(2′-hydroxyphenyl)-6-methoxy-4H-1-benzopyran-4-one) (FIG. 9C), and the physical and chemical properties thereof were as follows.

(1) Molecular formula: C₁₆H₁₂O₅

(2) Molecular weight: 284, ESI-MS: m/z 283.0 [M−H]⁺, m/z 285.0 [M+H]⁺, m/z 306.9 [M+Na]⁺ (FIG. 8)

(3) Melting point: 185° C.

(4) Appearance: pale yellow powder

(5) Solubility: Soluble in methanol, ethanol, ethyl acetate, and chloroform, and insoluble in water

(6) TLC staining: FeCl₃ (positive), bromocresol green (negative), UV light (positive, yellowish green), aniline diphenylamine (negative), antimony (negative), Dragendorff (negative)

(7) Maximum UV absorption wavelength range (methanol, λmax, nm): 218, 286, 255sh, and 323, (+NaOAc): 251, 353, (+AlCl₃): 218, 251, 317, 353: (+NAOAC+H₃BO₃) 214, 263, 289SH, AND 338SH

(8) ¹H and ¹³C-NMR:

¹H NMR (600 MHz, DMSO-d6) δ ppm: 8.13 (1H, d, 2-H), 7.47 (1H, d, 5-H), 6.78 (1H, d, 8-H), 6.92 (1H, d, 3′-H), 7.24 (1H, m, 4′-H), 6.91 (1H, d, 5′-H), 7.24 (1H, s, 6′-H), 3.93 (3H, 6-OCH₃) (FIG. 9A)

¹³C-NMR (150 MHz, DMSO-d6) δ ppm: 157.0 (2-C), 124.0 (3-C), 179.3 (4-C), 105.2 (5-C), 151.9 (6-C), 162.3 (7-C), 105.2 (8-C), 157.0 (9-C), 116.2 (10-C), 123.2 (1′-C), 118.9 (3′-C), 131.9 (4′-C), 122.3 (5′-C), 132.7 (6′-C), 57.5 (OCH₃) (FIG. 9B, C).

(9)

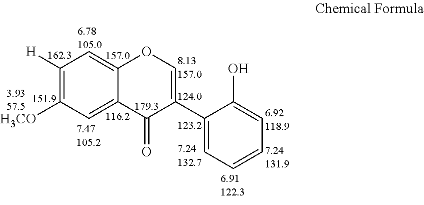 [SMILES: COc1ccc2occ(-c3ccccc3O)c(=O)c2c1]

Although specific embodiments of the present invention have been disclosed in detail as described above, it is obvious to those skilled in the art that such description is merely of preferable exemplary embodiments and is not construed to limit the scope of the present invention. Therefore, the substantial scope of the present invention will be defined by the appended claims and equivalents thereof.

### INDUSTRIAL APPLICABILITY

According to the present invention, the Salicornia SPP.-derived salty sauce contains an increased amount of a functional polyphenol compound having an antihypertensive effect. In particular, the salty sauce contains 5-deoxy-irilin B having very high ACE inhibition activity, and can thus be efficiently utilized as functional food for the prevention of hypertension and cerebrovascular disease, and includes large amounts of Salicornia SPP.-derived organic nutrients, such as minerals, amino acids, and enzymes, and is thus nutritionally useful.

1. A Salicornia SPP.-derived salty sauce, containing a 5-deoxy-irilin B (7-hydroxy-3-(2′-hydroxyphenyl)-6-methoxy-4H-1-benzopyran-4-one) compound represented by Chemical Formula 1 below.

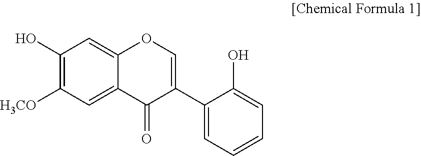 [SMILES: COc1cc2c(=O)c(-c3ccccc3O)coc2cc1O]

2. The Salicornia SPP.-derived salty sauce of claim 1, where a sodium chloride (NaCl) content is 6.0 to 39 wt % or a sodium content is 2.4 to 15.8 wt %, and a weight ratio of potassium (K) to sodium (Na) is 1:1 to 1:15.

3. A method of producing a Salicornia SPP.-derived salty sauce, comprising:
(a) washing Salicornia SPP.;
(b) cutting or grinding the washed Salicornia SPP.;
(c) hydrolyzing the cut or ground Salicornia SPP., thus obtaining a Salicornia SPP. hydrolysate;
(d) squeezing or extracting the Salicornia SPP. hydrolysate, thus obtaining a squeezed or extracted Salicornia SPP. liquid;
(e) purifying the squeezed or extracted Salicornia SPP. liquid, thus obtaining a purified Salicornia SPP. liquid; and
(f) concentrating the purified Salicornia SPP. liquid.

4. A method of producing a Salicornia SPP.-derived salty sauce, comprising:
(a) washing Salicornia SPP.;
(b) cutting or grinding the washed Salicornia SPP.;
(c) squeezing or extracting the cut or ground Salicornia SPP., thus obtaining a squeezed or extracted Salicornia SPP. liquid;
(d) purifying the squeezed or extracted Salicornia SPP. liquid, thus obtaining a purified Salicornia SPP. liquid; and
(e) concentrating the purified Salicornia SPP. liquid.

5. The method of claim 3, further comprising heating the cut or ground Salicornia SPP., after the cutting or grinding the washed Salicornia SPP.

6. The claim 3, wherein the hydrolyzing is performed using a biological process or a chemical process.

7. The method of claim 3, wherein the purifying is performed using at least one selected from the group consisting of a hydrophobic adsorbent, a cation exchange resin, and activated carbon.

8. The method of claim 3, wherein the concentrating the purified Salicornia SPP. liquid is performed so that the Salicornia SPP.-derived salty sauce has a solid content of 16 to 54 wt %.

9. A 5-deoxy-irilin B (7-hydroxy-3-(2′-hydroxyphenyl)-6-methoxy-4H-1-benzopyran-4-one) compound having angiotensin-I-converting enzyme (ACE) inhibition activity, represented by Chemical Formula 1 below.

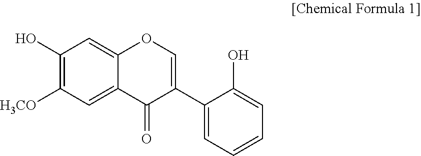 [SMILES: COc1cc2c(=O)c(-c3ccccc3O)coc2cc1O]

10. The 5-deoxy-irilin B (7-hydroxy-3-(2′-hydroxyphenyl)-6-methoxy-4H-1-benzopyran-4-one) compound of claim 9, wherein the compound of Chemical Formula 1 is separated from Salicornia SPP.

11. A composition for preventing and treating hypertension, containing, as an active ingredient, a 5-deoxy-irilin B (7-hydroxy-3-(2′-hydroxyphenyl)-6-methoxy-4H-1-benzopyran-4-one) compound, represented by Chemical Formula 1 below.

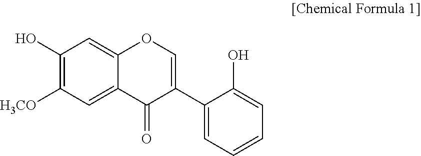 [SMILES: COc1cc2c(=O)c(-c3ccccc3O)coc2cc1O]

12. A method of separating a 5-deoxy-irilin B (7-hydroxy-3-(2′-hydroxyphenyl)-6-methoxy-4H-1-benzopyran-4-one) compound having angiotensin-I-converting enzyme (ACE) inhibition activity, represented by Chemical Formula 1 below, the method comprising subjecting a Salicornia SPP. extract to organic solvent fractionation, column chromatography purification, and high-performance liquid chromatography (HPLC).

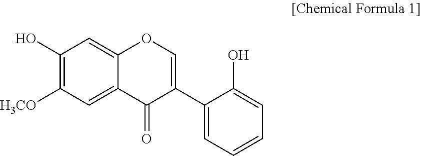 [SMILES: COc1cc2c(=O)c(-c3ccccc3O)coc2cc1O]

13. The method of claim 12, wherein the Salicornia SPP. extract is a methanol extract of a Salicornia SPP.-derived salty sauce obtained via hydrolysis using an enzyme.

14. The method of claim 4, further comprising heating the cut or ground Salicornia SPP., after the cutting or grinding the washed Salicornia SPP.

15. The method of claim 4, wherein the purifying is performed using at least one selected from the group consisting of a hydrophobic adsorbent, a cation exchange resin, and activated carbon.

16. The method of claim 4, wherein the concentrating the purified Salicornia SPP. liquid is performed so that the Salicornia SPP.-derived salty sauce has a solid content of 16 to 54 wt %.
